(19) United States
(12) Patent Application Publication
(10) Pub. No.: US 2009/0069897 A1
(43) Pub. Date: Mar. 12, 2009

(54) Spinal Interbody Device

(76) Inventors: Michael S. Butler, St. Charles, IL (US); Brian D. Hartsell, Aurora, IL (US)

Correspondence Address: Bruce J. Bowman;Bowman & Davis, P.A., Suite 106, 1016 3rd Ave. SW, Carmel, IN, 46032, US

(21) Appl. No.: 12/209,884

(22) Filed: Sep. 12, 2008

Related U.S. Application Data
(60) Provisional application No. 60/993,476, filed on Sep. 12, 2007.

(51) Int. Cl. A61F 2/44

(52) U.S. Cl. 623/17.16; 623/17.11

(57) ABSTRACT

A spinal interbody or intravertebral device is provided for fusion and non-fusion spinal applications. The spinal interbody device allows for limited motion between adjacent vertebrae to help preserve normal motion between adjacent vertebrae (superior/inferior vertebrae) relative to the implanted spinal interbody device and limited rotational motion of the spinal interbody device, once implanted. Limitation of motion is achieved through one or more protrusions incorporated into the spinal interbody device. The protrusions preferably, but not necessarily, extend radially from a hub or ball-shaped body of the interbody device. In one form, a single protrusion extends radially about the hub of the interbody device to form a continuous band, ring or base about the hub. In this form, portions of the band may extend radially further from the hub than other portions of the band. Preferably, but not necessarily, in this form the band extends radially a greater distance from the hub in the posterior and anterior directions. In another form, a first protrusion extends radially from one side of the hub while a second protrusion extends radially from another side of the hub. Anchoring means may also be provided for attachment to upper and lower vertebrae.

### RELATED APPLICATIONS

This patent application claims the benefit of and/or priority to U.S. Provisional Patent Application Ser. No. 60/993,476 filed Sep. 12, 2007, entitled “Spinal Interbody Device” the entire contents of which is specifically incorporated herein by this reference.

### BACKGROUND OF THE INVENTION

1. Field of the Invention

The present invention relates to spinal interbody devices for implantation between a pair of adjacent vertebrae in order to provide support to the adjacent vertebrae and/or promote bone fusion between the adjacent vertebrae.

2. Background Information

The disc between vertebrae of a human spine is sometimes damaged due to disease or injury, or may simply deteriorate due to age, disease, injury or congenital defect. With others, the vertebrae may become compressed or otherwise damaged. In these and other cases the vertebrae can become too closely spaced anteriorly which causes an undesired abnormal curvature of the spine with respect to lordosis or kyphosis.

Because of this, surgery may be utilized to place one or more spinal spacers or interbody devices between adjacent vertebrae in order to provide proper spacing of the vertebrae. In some cases the spinal interbody device may be an artificial or prosthetic spinal disc that is designed to replace the existing spinal disc. In some cases the spinal interbody device is a device that supports and/or realigns the adjacent vertebrae relative to one another and that promotes fusion between the vertebrae. Typically, bone fusion material is often placed about or in the spinal interbody device in order to promote growth of the bone between the adjacent vertebrae and thereby create fusion of the adjacent vertebrae.

When spinal interbody devices are used, it is desirable for them to engage as much surface of the bone of the vertebrae as possible to provide support to the bone and to thereby reduce the likelihood of subsidence of the device into the vertebrae or bone resulting from contact pressure of the spinal interbody device against bone surfaces. Subsidence can occur since part of the bone is somewhat spongy in nature, especially near the centers of the adjacent vertebrae.

The structure of spinal interbody devices functions to support the two adjacent vertebral surfaces, unless the spinal interbody device is also used as a fusion cage within or around which to pack bone fusion material. Because it is desirable in such structures to maintain weight and volume as low as possible in order to make the device more compatible with the body, it is also desirable to make the spinal interbody device as small and lightweight as possible, while still maintaining strength. One problem with these spinal interbody devices, however, is that they do not provide for any preservation of motion for the user. Moreover, these spinal interbody devices are deficient in other areas.

Accordingly, there presently exists a need for an improved spinal interbody device.

### SUMMARY OF THE INVENTION

A spinal interbody or intravertebral device is disclosed for fusion and non-fusion spinal applications. The spinal interbody device allows for limited motion between adjacent vertebrae to help preserve normal motion between adjacent vertebrae (superior/inferior vertebrae) relative to the implanted spinal interbody device. Rotational motion of the spinal interbody device, once implanted, may also be limited by the present configuration.

The present spinal interbody device is configured to provide a limitation on motion between adjacent vertebrae. This limitation of spinal motion is particularly with respect to an inferior/superior axis of the spine. Such motion limitation is achieved through one or more protrusions incorporated into the spinal interbody device. The protrusions preferably, but not necessarily, extend radially from a hub or ball-shaped body of the interbody device.

In one form, a single protrusion extends radially about the hub of the interbody device to form a continuous band, ring or base about the hub. In this form, portions of the band may extend radially further from the hub than other portions of the band. This provides a platform whose dimensions limit the motion between adjacent vertebrae. Preferably, but not necessarily, in this form the band extends radially a greater distance from the hub in the posterior and anterior directions.

In another form, a first protrusion extends radially from one side of the hub while a second protrusion extends radially from another side of the hub. In this form, the first and second protrusions provide a platform whose dimensions limit the motion between adjacent vertebrae.

The spinal interbody device is preferably, but not necessarily, formed of a pliant or soft hub or ball-shaped body with a base or protrusions. The base or protrusions extend from the hug in both the superior and inferior directions. The pliant hub is formed by a first pliant dome extending from the base in the superior direction and a second pliant dome extending from the base in the inferior direction.

In one form, the base or protrusion(s) are rigid. In this form, the protrusion(s) is (are) preferably, but not necessarily, formed of PEEK (polyetheretherketone) but may be formed from other biocompatible thermoplastics, polymers, metals or materials as desired. In another form, the base is a pliant and may be formed of various biocompatible thermoplastics, polymers, metals or materials as desired. The pliant or soft hub is preferably, but not necessarily, formed of a biocompatible thermoplastic but may be formed from other biocompatible thermoplastics, polymers, metals or materials as desired.

The base may be solid or may have one or more cavities therein that may be used for bone fusion material. The base may additionally or separately have a bore therein or therethrough for bone fusion purposes. The base is shaped to be received between adjacent vertebrae and in one form is rectangular.

In all forms, the spinal interbody device may include one or more fastening, attaching or anchoring devices for holding the spinal interbody device in place, and also serving as anti-back-out devices to ensure that the device does not back out through the same path that it was installed through. Such devices may include staples (having one or more spikes per staple), spikes or the like with barbs, hooks, flanges or the like to prevent the fastener from pulling out of the vertebral bone.

### BRIEF DESCRIPTION OF THE DRAWINGS

The above mentioned and other features, advantages and objects of this invention, and the manner of attaining them, will become apparent and the invention itself will be better understood by reference to the following description of embodiments of the invention taken in conjunction with the accompanying drawings, wherein:

FIG. 1 is a posterior perspective view of a spinal interbody device fashioned in accordance with the present principles;

FIG. 2 is a side view of the spinal interbody device of FIG. 1;

FIG. 3 is a bottom view of the spinal interbody device of FIG. 1; and

FIG. 4 is a posterior perspective view of another embodiment of a spinal interbody device fashioned in accordance with the present principles.

Like reference numerals indicate the same or similar parts throughout the several figures.

A detail of the features, functions and/or configuration of the components depicted in the various figures will now be presented. It should be appreciated that not all of the features of the components of the figures are necessarily described. Some of these non discussed features as well as discussed features are inherent from the figures. Other non discussed features may be inherent in component geometry and/or configuration.

### DETAILED DESCRIPTION OF EMBODIMENTS OF THE INVENTION

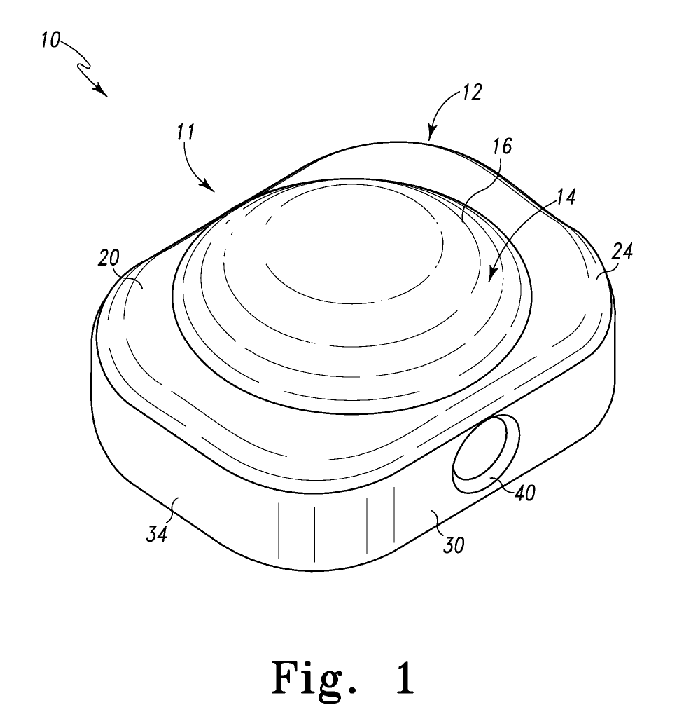

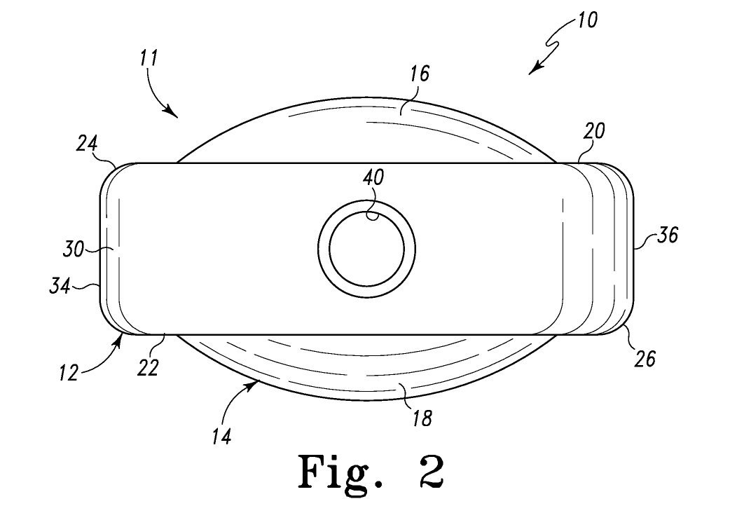

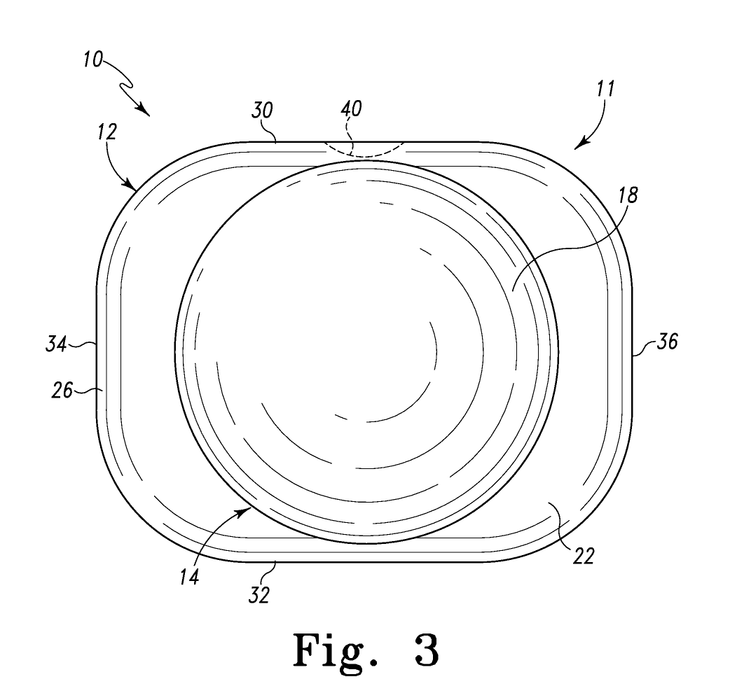

Referring to FIGS. 1-3 there is depicted an exemplary spinal interbody or intravertebral device, generally designated 10, fashioned in accordance with the present principles. The spinal interbody device 10 is characterized by a body 11 having a base, ring, band or a protrusion 12 and a ball-shaped or rounded hub, core, middle or center 14. The body 11 is sized to be received between adjacent vertebrae of the spine but to not extend beyond the periphery of the vertebra. As such, the body 11 may be fabricated in various sizes to accommodate various sizes of vertebrae and/or applications.

The base 12 may be solid (as shown) or may have one or more cavities therein. The one or more cavities may be in communication with an exterior of the body through a bore or channel in the body. Alternatively or additionally, the body may have one or more bores or channels therethrough. For instance, the body may have a single hole or bore through the center or middle of the base, or may have two intersecting holes or bores therethrough. Various combinations are contemplated.

The base 12 may be rigid or may be pliant (soft). In the rigid form, the base is preferably, but not necessarily, formed of PEEK (polyetheretherketone) but may be formed from other biocompatible thermoplastics, polymers, metals or materials as desired. In the soft or pliant form, the base may be formed of various biocompatible thermoplastics, polymers, metals or materials as desired. Moreover, while the base is shown as being rectangular, the base may take other shapes as desired and/or is appropriate.

The hub 14 may be rigid or may be pliant (soft). In the rigid form, the hub is preferably, but not necessarily, formed from PEEK (polyetheretherketone) but may be formed from other biocompatible thermoplastics, polymers, metals or materials as desired. It should be appreciated that the hub may be formed from other biocompatible thermoplastics, polymers or materials (e.g. metals), as desired and/or is appropriate.

The base 12 defines a superior end, side or surface 20 and an inferior end, side or surface 22, a posterior end, side or surface 30 and an anterior end, side or surface 32, and a first lateral side 34 and a second lateral side 36.

A bore, cavity or depression 40 is formed in the posterior end 30 of the base 12 and is configured to accept a tool for implanting and/or adjusting the device 10. It should be appreciated that the bore, cavity or depression 40 may instead be formed in another end, side or surface of the base 12. Moreover, the bore, cavity or depression may likewise be formed in one or more additional ends, sides or surfaces of the base 12.

The hub 14 is made from a biocompatible material in like manner to the base 12. In one form, the hub 14 is made from the same material as the base 12 and thus has the same characteristics or features. In another form, the hub 14 is made from a material that has one or more characteristics or properties that are different than the base. In one form the hub 14 is formed of a material that is softer than the material of the base. In one form, the hub 14 is formed of a material that is pliant. Other characteristics or features of the hub 14 are contemplated and are not mutually exclusive. In the preferred form, the hub 14 is formed of a biocompatible thermoplastic but may be formed from other biocompatible thermoplastics, polymers, metals or materials as desired. The hub 14 is defined by a first dome portion 16 that extends from the superior surface 20 of the base 12 and a second dome portion 18 that extends from the inferior surface 22 of the base 12 (the nomenclature first and second being arbitrary). When the body 11 is implanted, the dome portion 16 is configured to extend up into a lower portion of a superior vertebra of adjacent vertebrae while the dome portion 18 is configured to extend down into an upper portion of an inferior vertebra of the adjacent vertebrae.

When implanted, the base 12 (i.e. the superior or inferior surface of the base) provides a seating surface or platform for the bone of an upper or lower adjacent vertebrae. The hub 14 and particularly the dome portion, allows the vertebrae to rotate thereabout. The dome portion is situated centrally in the upper or lower portion of the vertebrae as appropriate. The thickness and/or other dimensions of the platform allows for limited motion and rotational stability.

The hub 14 may be formed as separate dome portions over-molded onto the rectangular base 12. Alternatively, the hub 14 may be formed as one piece molded into the base 12. Still further, the hub 14 may formed as one piece or integral with the base 14. In this regard the device may be an integrally molded, all PEEK design. Other configurations are contemplated.

Rather than have a relatively flat superior surface 20, the superior surface 20 of the base 12 may be angled or have/provide angulation. Such angulation may be lordotic and/or kyphotic angulation. Likewise, rather than have a relatively flat inferior surface 22, the inferior surface 22 of the base 12 may be angled or have/provide angulation. Such angulation may be lordotic and/or kyphotic angulation and may be in addition to or separate from any superior surface angulation.

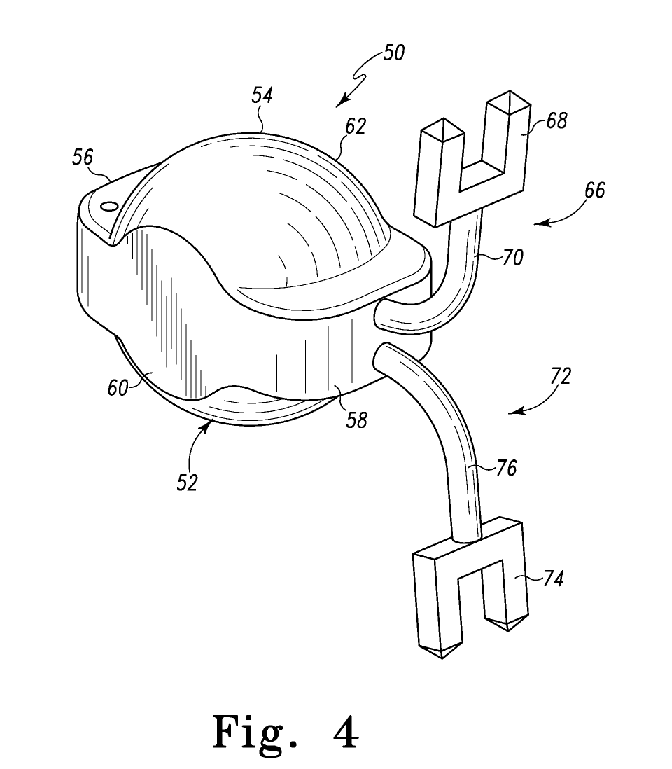

Referring to FIG. 4, there is depicted another exemplary spinal interbody or intravertebral device, generally designated 50, fashioned in accordance with the present principles. The spinal interbody device 50 is characterized by a body 52 having a ball-shaped or rounded hub, core, middle or center 54 and first and second protrusions, flanges or ledges 56 and 58 that extend radially from the hub 54. As shown, the first protrusion 56 extends radially from the anterior side of the hub 54 while the second protrusion 58 extends radially from the posterior side of the hub 54. The body 52 is sized to be received between adjacent vertebrae of the spine but to not extend beyond the periphery of the vertebra. As such, the body 52 may be fabricated in various sizes to accommodate various sizes of vertebrae and/or applications.

The protrusions 56, 58 may be solid (as shown) or may have one or more cavities therein. The one or more cavities may be in communication with an exterior of the body through a bore or channel in the body. Alternatively or additionally, the body may have one or more bores or channels therethrough. For instance, the body may have a single hole or bore through the center or middle of the base, or may have two intersecting holes or bores therethrough. Various combinations are contemplated.

The hub 54 may be rigid or may be pliant (soft). In the rigid form, the hub is preferably, but not necessarily, formed of PEEK (polyetheretherketone) but may be formed from other biocompatible thermoplastics, polymers, metals or materials as desired. In the soft or pliant form, the hub may be formed of various biocompatible thermoplastics, polymers, metals or materials as desired.

The protrusions 56, 58 of the embodiment shown in the figures is preferably, but not necessarily, made from PEEK (polyetheretherketone) or other biocompatible thermoplastic. However, it should be appreciated that the protrusions may be formed from other biocompatible thermoplastics, polymers or materials (e.g. metals), as desired and/or is appropriate. These may be rigid or pliant. Moreover, while the protrusions are shown as being generally rectangular, they may take other shapes as desired and/or is appropriate.

The protrusions 56, 58 each define a superior end, side or surface and an inferior end, side or surface, while the protrusion 56 is on an anterior side of the hub 54 and the protrusion 58 is on a posterior side of the hub 54. The hub 54 has a first lateral side 60 and a second lateral side 62. The first lateral side 60 is generally flat or planar. Likewise, the second lateral side 62 is generally flat or planar.

The hub 54 is made from a biocompatible material in like manner to the protrusions 56, 58. In one form, the hub 54 is made from the same material as the protrusions 56, 58 and thus has the same characteristics or features. In another form, the hub 54 is made from a material that has one or more characteristics or properties that are different than the protrusions. In one form the hub 54 is formed of a material that is softer than the material of the protrusions. In one form, the hub 54 is formed of a material that is pliant. Other characteristics or features of the hub 54 are contemplated and are not mutually exclusive. In the preferred form, the hub 54 is formed of a biocompatible thermoplastic but may be formed from other biocompatible thermoplastics, polymers, metals or materials as desired. The hub 54 is defined by a first dome portion that extends from the superior side of the hub 54 and a second dome portion that extends from the inferior side of the hub 54 (the nomenclature first and second being arbitrary). When the device 50 is implanted, one dome portion is configured to extend up into a lower portion of a superior vertebra of adjacent vertebrae while the other dome portion is configured to extend down into an upper portion of an inferior vertebra of the adjacent vertebrae.

When implanted, the protrusions 56, 58 (i.e. the superior or inferior surface of the base) provides a seating surface, platform for the bone of an upper or lower adjacent vertebrae. The hub 54 and particularly the dome portion, allows the vertebrae to rotate thereabout. The dome portion is situated centrally in the upper or lower portion of the vertebrae as appropriate.

The hub 54 and the protrusions may be formed as a single piece. Alternatively, the hub 54 may be formed as multiple pieces molded together. In this regard the device may be an integrally molded, all PEEK design. Other configurations are contemplated.

The spinal interbody device 50 further includes one or more fasteners, attachments, fastening, attaching and/or anchoring devices for holding the spinal interbody device 50 in place, and also serving as anti-back-out devices to ensure that the device does not back out through the same path that it was installed through.

The spinal interbody device 50 is shown having a first fastener 66 comprises of a two-pronged staple 68 attached via a tether 70 to the end of the protrusion 58. The prongs may or may not be barbed. A second fastener 72 is also provided comprising a two-pronged staple 74 attached via a tether 76 to the end of the protrusion 58. Of course, other devices may be used including staples (having one or more spikes per staple), spikes or the like with barbs, hooks, flanges or the like. The staple 68 is designed to be received by an endplate of one vertebral body (vertebra) while the staple 74 is designed to be received by an endplate of another vertebral body (vertebra) adjacent the other vertebra between which the spinal interbody device 50 is implanted.

It should be appreciated that one or more fasteners may be provided on the end of the protrusion 56 in like manner to that shown with respect to protrusion 58. Moreover, a single tether may extend through a superior/inferior bore in the device (such as the one seen in the protrusion 56 of the spinal interbody device 50) with the tether have a staple or the like on both ends thereof.

Rather than have a relatively flat superior surface, the superior surface of the protrusions may be angled or have/provide angulation. Such angulation may be lordotic and/or kyphotic angulation. Likewise, rather than have a relatively flat inferior surface, the inferior surface of the protrusions may be angled or have/provide angulation. Such angulation may be lordotic and/or kyphotic angulation and may be in addition to or separate from any superior surface angulation.

While the invention has been illustrated and described in detail in the drawings and foregoing description, the same is to be considered as illustrative and not restrictive in character, it being understood that only preferred embodiments have been shown and described and that all changes and modifications that come within the spirit of the invention are desired to be protected.

What is claimed is:

1. A spinal interbody device comprising:
a rounded hub defining a superior portion and an inferior portion; and
a base extending radially outwardly from the rounded hub and disposed between the superior portion and the inferior portion.

2. The spinal interbody device of claim 1, wherein the base defines a platform extending about a circumference of the rounded hub.

3. The spinal interbody device of claim 2, wherein the platform extends radially a first distance from first and second lateral sides of the rounded hub and radially from a second distance from the posterior and anterior ends.

4. The spinal interbody device of claim 3, wherein the second distance is greater than the first distance.

5. The spinal interbody device of claim 4, wherein the rounded hub is formed of a pliant biocompatible material and the base is formed of a rigid biocompatible material.

6. The spinal interbody device of claim 4, wherein the pliant biocompatible material is adjustable in size while in situ.

7. The spinal interbody device of claim 4, wherein the rounded hub and the base are formed of a rigid material.

8. The spinal interbody device of claim 4, wherein the rigid material comprises PEEK.

9. A spinal interbody device comprising:
a rounded hub;
a first protrusion extending radially from a posterior end of the rounded hub; and
a second protrusion extending radially from an anterior end of the rounded hub.

10. The spinal interbody device of claim 9, further comprising:
a first flat disposed on a first lateral side of the rounded hub; and
a second flat disposed on a second lateral side of the rounded hub.

11. The spinal interbody device of claim 9, wherein:
the first protrusion is generally rectangular and defines a first platform;
and the second protrusion is generally rectangular and defines a second platform.

12. The spinal interbody device of claim 11, wherein the rounded hub is formed of a pliant biocompatible material and the first and second protrusions are formed of a rigid biocompatible material

13. The spinal interbody device of claim 12, wherein the pliant biocompatible material is adjustable in size while in situ.

14. The spinal interbody device of claim 11, wherein the rounded hub and the first and second protrusions are formed of a rigid biocompatible material.

15. The spinal interbody device of claim 14, wherein the rigid material comprises PEEK.

16. The spinal interbody device of claim 11, further comprising a fastener extending from the first protrusion.

17. The spinal interbody device of claim 16, wherein the fastener comprises a bone anchor attached to a tether that extends from the first protrusion.

18. The spinal interbody device of claim 16, wherein the fastener comprises a first bone anchor attached to a first tether that extends from the first protrusion and a second bone anchor attached to a second tether that extends from the first protrusion.

19. The spinal interbody device of claim 18, wherein the first bone anchor comprises a first staple and the second bone anchor comprises a second staple.

20. The spinal interbody device of claim 16, wherein the fastener comprises a tether extending through a superior/inferior bore of the device, a first bone anchor attached to one end of the tether, and a second bone anchor attached to another end of the tether.

21. The spinal interbody device of claim 20, wherein the superior/inferior bore extends through one of the first and second protrusions.

22. A spinal interbody device comprising:
a rounded hub formed of a rigid biocompatible material;
a first generally rectangular protrusion extending radially from a posterior side of the rounded hub and formed of the rigid biocompatible material; and
a second generally rectangular protrusion extending radially from an anterior side of the rounded hub and formed of the rigid biocompatible material.

23. The spinal interbody device of claim 22, wherein the rigid biocompatible material is PEEK.

24. The spinal interbody device of claim 23, further comprising:
a first flat disposed on a first lateral side of the rounded hub; and
a second flat disposed on a second lateral side of the rounded hub.

25. The spinal interbody device of claim 22, further comprising a fastener extending from the first generally rectangular protrusion.

26. The spinal interbody device of claim 25, wherein the fastener comprises a bone anchor attached to a tether that extends from the first generally rectangular protrusion.

27. The spinal interbody device of claim 25, wherein the fastener comprises a first bone anchor attached to a first tether that extends from the first generally rectangular protrusion and a second bone anchor attached to a second tether that extends from the first generally rectangular protrusion.

28. The spinal interbody device of claim 27, wherein the first bone anchor comprises a first staple and the second bone anchor comprises a second staple.

29. The spinal interbody device of claim 25, wherein the fastener comprises a tether extending through a superior/inferior bore of the device, a first bone anchor attached to one end of the tether, and a second bone anchor attached to another end of the tether.

30. The spinal interbody device of claim 25, wherein the fastener comprises a tether having a plurality of bone anchors attached thereto.
